(19) United States
(12) Patent Application Publication
(10) Pub. No.: US 2024/0242787 A1
(43) Pub. Date: Jul. 18, 2024

(54) METHOD, PROGRAM, APPARATUS, AND SYSTEM FOR SEARCHING LOW MOLECULAR WEIGHT COMPOUND

(71) Applicant: DAIKIN INDUSTRIES, LTD., Osaka (JP)

(72) Inventor: Shohei TACHIKAWA, Osaka (JP)

(73) Assignee: DAIKIN INDUSTRIES, LTD., Osaka (JP)

(21) Appl. No.: 18/622,044

(22) Filed: Mar. 29, 2024

Related U.S. Application Data
(63) Continuation of application No. PCT/JP22/36775, filed on Sep. 30, 2022.

(30) Foreign Application Priority Data
Oct. 4, 2021 (JP) 2021-163292

(51) Int. Cl. G16C 20/40; G16C 20/30

(52) U.S. Cl. CPC G16C 20/40; G16C 20/30

(57) ABSTRACT

A method executed by a computer, the method including acquiring a chemical structure of a plurality of low molecular weight compounds; calculating an estimate value of a physical property value of each of the low molecular weight compounds and a variation of the estimate values, from the acquired chemical structure of each of the low molecular weight compounds, by using a regression model; and calculating an acquisition function from the estimate values of the physical property value and the variation of the estimate values.

### CROSS-REFERENCE TO RELATED APPLICATION

The present application is a continuation application of International Application No. PCT/JP2022/036775 filed on Sep. 30, 2022, which is based on and claims priority to Japanese Patent Application No. 2021-163292 filed on Oct. 4, 2021. The contents of these applications are incorporated herein by reference in their entirety.

### TECHNICAL FIELD

The present disclosure relates to a method, a program, an apparatus, and a system for searching a low molecular weight compound.

### BACKGROUND ART

Conventionally, in order to search for a material that achieves a desired function, characteristic, or physical property, an experimenter has had to conduct an experiment based on the knowledge and experience of the experimenter. However, in such a material searching method, the success of the material searching is affected by the knowledge and experience of the experimenter.

For this reason, in recent years, a technique referred to as materials informatics, which uses information science to search for materials that achieve the desired function, characteristic, and physical property, is now known (Patent Document 1).

### RELATED ART DOCUMENTS

### Patent Documents

[Patent Document 1] Japanese Unexamined Patent Application Publication No. 2020-71827

However, in Patent Document 1, the target of the search is a polymer (a polymer compound), and is not a low molecular weight compound. In the present disclosure, an object is to search for a low molecular weight compound having a high possibility of having a desired physical property.

### SUMMARY

The method according to the first aspect of the present disclosure is:
a method executed by a computer, the method including acquiring a chemical structure of a plurality of low molecular weight compounds;
calculating an estimate value of a physical property value of each of the low molecular weight compounds and a variation of the estimate values, from the acquired chemical structure of each of the low molecular weight compounds, by using a regression model; and
calculating an acquisition function from the estimate values of the physical property value and the variation of the estimate values.

A second aspect of the present disclosure is the method described in the first aspect, further including:
determining the low molecular weight compound for which an expected improvement is maximum, in a case where the acquisition function is the expected improvement.

Further, the third aspect of the present disclosure is a method described in the first aspect, further including:
selecting at least one of the low molecular weight compounds from among the plurality of the low molecular weight compounds; and
generating the regression model by using the chemical structure of the selected at least one of the low molecular weight compounds and a measured value of the physical property value of the selected at least one of the low molecular weight compounds.

Further, a fourth aspect of the present disclosure is a method described in a first aspect, further including:
replacing the chemical structures of the plurality of the low molecular weight compounds with a plurality of types of descriptors;
selecting at least one of the low molecular weight compounds from among the plurality of the low molecular weight compounds based on the plurality of types of descriptors; and
generating the regression model by using the chemical structure of the selected at least one of the low molecular weight compounds and a measured value of the physical property value of the selected at least one of the low molecular weight compounds.

A fifth aspect of the present disclosure is a method described in a fourth aspect, further including:
converting the chemical structures of the plurality of the low molecular weight compounds into character strings.

Further, a sixth aspect of the present disclosure is a method described in a fourth aspect, further including:
replacing the chemical structures of the plurality of the low molecular weight compounds with a plurality of types of RDKit2D descriptors.

A seventh aspect of the present disclosure is a method described the first aspect, wherein:
the estimate value is an average value of a probability distribution, and the variation is a standard deviation of the probability distribution.

Further, the eighth aspect of the present disclosure is a method described in the first aspect, wherein:
the plurality of the low molecular weight compounds include at least one of a compound selected by a human, a compound extracted from a database, or a compound designed by a predetermined algorithm.

A ninth aspect of the present disclosure is a method described in the first aspect, wherein:
the regression model is a Gaussian process regression model.

Further, the tenth aspect of the present disclosure is a method described in any of the first aspect, further including:
correcting the estimate value of the physical property value; and
generating the regression model by using the chemical structure of the low molecular weight compound for which the estimate value of the physical property value has been corrected, and the corrected estimate value of the physical property value.

The method according to an eleventh aspect of the present disclosure is:
a method executed by a computer, the method including:
replacing a chemical structure of a plurality of low molecular weight compounds with a plurality of types of descriptors; and
selecting a most diverse group of compounds from among the plurality of the low molecular weight compounds, by selecting a combination in which det (X^{T}X) is maximum for the plurality of types of descriptors.

Further, a twelfth aspect of the present disclosure is a method described in the eleventh aspect, further including:
selecting, with respect to the plurality of types of the descriptors, at least one of the descriptors from among highly correlated descriptors, wherein
the most diverse group of compounds is selected by using the selected at least one of the descriptors, and
a number of the selected at least one of the descriptors is less than or equal to a number of the low molecular weight compounds forming the most diverse group of compounds.

Further, a thirteenth aspect of the present disclosure is a method described in the eleventh aspect, wherein:
the most diverse group of compounds is selected by using a genetic algorithm.

Further, a fourteenth aspect of the present disclosure is a method described in an eleventh aspect, further including:
generating a regression model by using the chemical structure of the low molecular weight compounds forming the most diverse group of compounds and a measured value of a physical property value of the low molecular weight compounds forming the most diverse group.

A non-transitory computer-readable recording medium according to a fifteenth aspect of the present disclosure is:
a non-transitory computer-readable recording medium storing a program that causes a computer to execute a process, the process including:
acquiring a chemical structure of a plurality of low molecular weight compounds;
calculating an estimate value of a physical property value of each of the low molecular weight compounds and a variation of the estimate values, from the acquired chemical structure of each of the low molecular weight compounds, by using a regression model; and
calculating an acquisition function from the estimate values of the physical property value and the variation of the estimate values.

An apparatus according to the sixteenth aspect of the present disclosure is
an apparatus including:
a processor; and
a memory that includes instructions, which when executed, cause the processor to execute:
acquiring a chemical structure of a plurality of low molecular weight compounds;
calculating an estimate value of a physical property value of each of the low molecular weight compounds and a variation of the estimate values, from the acquired chemical structure of each of the low molecular weight compounds, by using a regression model; and
calculating an acquisition function from the estimate values of the physical property value and the variation of the estimate values.

A system according to a seventeenth aspect of the present disclosure is
a system including:
a server; and
a user terminal, wherein
the server includes:
a processor; and
a memory that includes instructions, which when executed, cause the processor to execute:
acquiring a chemical structure of a plurality of low molecular weight compounds;
calculating an estimate value of a physical property value of each of the low molecular weight compounds and a variation of the estimate values, from the acquired chemical structure of each of the low molecular weight compounds, by using a regression model;
calculating an acquisition function from the estimate values of the physical property value and the variation of the estimate values; and
presenting the acquisition function to the user terminal.

### BRIEF DESCRIPTION OF DRAWINGS

FIG. 1 is a diagram illustrating an overall configuration according to an embodiment of the present disclosure;

FIG. 2 is a diagram illustrating a hardware configuration of a compound search apparatus according to an embodiment of the present disclosure;

FIG. 3 is a diagram for explaining the overall flow according to an embodiment of the present disclosure;

FIG. 4 is a diagram for explaining the overall flow according to an embodiment of the present disclosure;

FIG. 5 is a diagram for explaining the substitution of chemical structures with a plurality of types of descriptors according to an embodiment of the present disclosure; and

FIG. 6 is a diagram for explaining the selection of the most diverse group of compounds according to an embodiment of the present disclosure.

### DESCRIPTION OF EMBODIMENTS

Embodiments of the present disclosure will be described below based on the drawings.

### Terminology

As used herein, a “low molecular weight compound” is a compound having a molecular weight of 10,000 or less.

### <Overall Configuration>

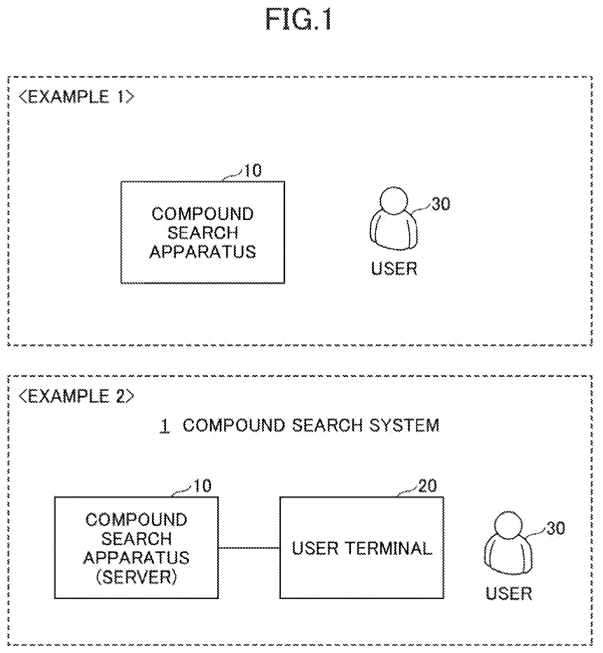

FIG. 1 is a diagram illustrating an overall configuration according to an embodiment of the present disclosure. Hereinafter, the description will be divided into Example 1 and Example 2.

### Example 1

As illustrated in the upper part of FIG. 1, in Example 1, a user 30 can operate a compound search apparatus 10 to have the compound search apparatus 10 search for a compound.

The compound search apparatus 10 performs a process of searching for a compound. Specifically, the compound search apparatus 10 receives an instruction input by the user 30, performs a process for searching for a compound, and presents the result of the search process. The compound search apparatus 10 is, for example, a personal computer, a tablet, a smartphone, etc.

### Example 2

As illustrated in the lower part of FIG. 1, in Example 2, the user 30 can operate a user terminal 20 to have the compound search apparatus 10 (for example, a server) search for a compound. A compound search system 1 includes the compound search apparatus 10 (for example, a server) and the user terminal 20.

The compound search apparatus 10 (for example, a server) performs a process of searching for a compound. Specifically, the compound search apparatus 10 (for example, a server) performs a process of searching for a compound in response to a request from the user terminal 20, and causes the user terminal 20 to present the result of the search process. The compound search apparatus 10 (for example, a server) includes one or more computers. The compound search apparatus 10 (for example, a server) can transmit and receive data to and from the user terminal 20 via any network.

The user terminal 20 requests the compound search apparatus 10 (for example, a server) to perform a process of searching for a compound and presents the result of the search. Specifically, the user terminal 20 transmits an instruction input by the user 30 to the compound search apparatus 10 (for example, a server). The user terminal 20 receives and presents the result of the process of searching for the compound from the compound search apparatus 10 (for example, a server). The user terminal 20 is, for example, a personal computer, a tablet, a smartphone, etc. The user terminal 20 can transmit and receive data to and from the compound search apparatus 10 (for example, a server) via any network.

### <Hardware Configuration>

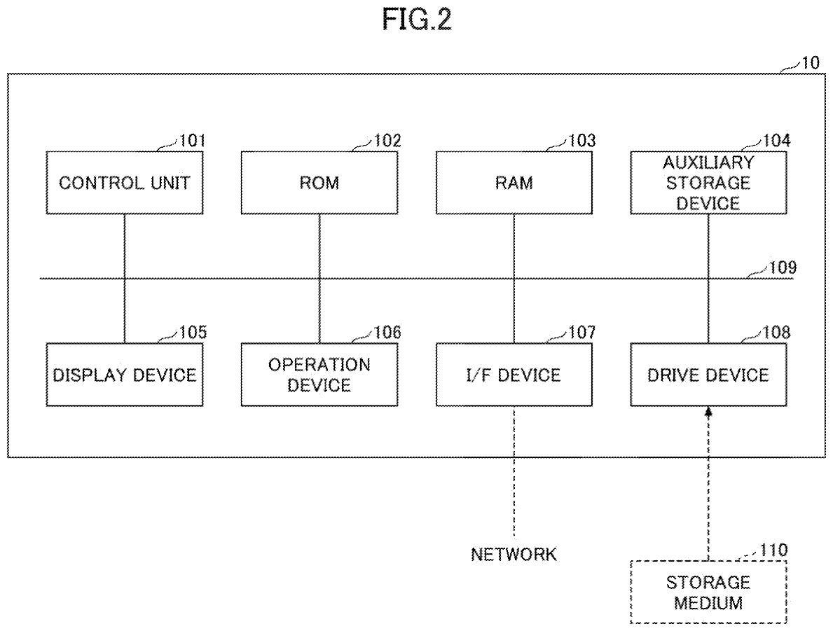

FIG. 2 is a diagram illustrating a hardware configuration of the compound search apparatus 10 according to an embodiment of the present disclosure. The same applies to the user terminal 20.

The compound search apparatus 10 includes a control unit 101, a ROM (Read Only Memory) 102, and a RAM (Random Access Memory) 103. The control unit 101, the ROM 102, and the RAM 103 form what is referred to as a computer. The compound search apparatus 10 may also include an auxiliary storage device 104, a display device 105, an operation device 106, an I/F (interface) device 107, and a drive device 108. The pieces of hardware of the compound search apparatus 10 are connected to each other via a bus 109.

The control unit (for example, a CPU) 101 is an arithmetic device for executing various programs installed in the auxiliary storage device 104. When the control unit 101 executes the programs, the processes described herein are executed.

The ROM 102 is a nonvolatile memory. The ROM 102 functions as a main storage device for storing various programs, data, etc., necessary for the control unit 101 to execute various programs installed in the auxiliary storage device 104. Specifically, the ROM 102 functions as a main storage device for storing boot programs such as BIOS (Basic Input/Output System) and EFI (Extensible Firmware Interface).

The RAM 103 is a volatile memory such as DRAM (Dynamic Random Access Memory) or SRAM (Static Random Access Memory). The RAM 103 functions as a main storage device that provides a work area that is expanded when various programs installed in the auxiliary storage device 104 are executed by the control unit 101.

The auxiliary storage device 104 is an auxiliary storage device that stores various programs and information used when various programs are executed.

The display device 105 is a display device that displays the internal state or the like of the compound search apparatus 10.

The operation device 106 is an input device to which an operator of the compound search apparatus 10 inputs various instructions to the compound search apparatus 10.

The I/F device 107 is a communication device for connecting to a network and communicating with other devices.

The drive device 108 is a device for setting a storage medium 110. The storage medium 110 includes a medium for recording information optically, electrically, or magnetically, such as a CD-ROM, a flexible disk, a magneto-optical disk or the like. The storage medium 110 may include a semiconductor memory or the like for recording information electrically, such as a ROM, a flash memory or the like.

The various programs installed in the auxiliary storage device 104 are installed, for example, when the distributed storage medium 110 is set in the drive device 108 and the various programs recorded in the storage medium 110 are read out by the drive device 108. Alternatively, the various programs installed in the auxiliary storage device 104 may be downloaded from the network via the I/F device 107 to be installed.

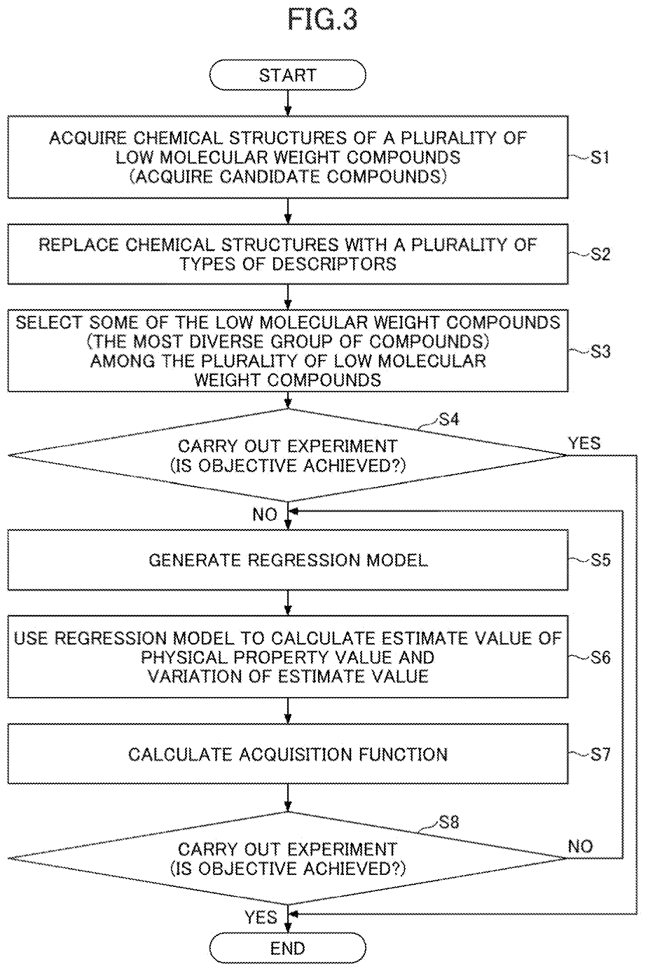

The process executed by the control unit 101 will be described below. The entire flow will be described with reference to FIG. 3, and then the details of each processing will be described.

### <Overview of the Entire Process>

FIG. 3 is a diagram for explaining the entire process according to an embodiment of the present disclosure. Note that the compound search apparatus 10 acquires a target value (for example, a target value entered by the experimenter, etc.) of the physical property value of the low molecular weight compound.

In step 1 (S1), the compound search apparatus 10 acquires the chemical structures of a plurality of low molecular weight compounds. That is, the compound search apparatus 10 acquires candidate compounds which are compounds to be candidates.

In step 2 (S2), the compound search apparatus 10 replaces the chemical structures of the plurality of low molecular weight compounds acquired in S1 with a plurality of types of descriptors (for example, the RDKit2D descriptor).

In step 3 (S3), the compound search apparatus 10 selects some of the low molecular weight compounds (for example, the most diverse group of compounds) among the plurality of low molecular weight compounds acquired in S1, based on the plurality of types of descriptors obtained in S2. Specifically, the compound search apparatus 10 selects the most diverse group of compounds by using the D-optimization technique.

In step 4 (S4), the experimenter carries out an experiment by using the most diverse group of compounds selected in S3. As a result of the experiment, if the objective is achieved (if the measured value of the physical property value satisfies the target value of the physical property value), the process is ended, and if the objective is not achieved, the process proceeds to step 5.

In step 5 (S5), the compound search apparatus 10 generates a regression model (specifically, a Gaussian process regression model) by using the most diverse group of compounds selected in S3. Specifically, the compound search apparatus 10 generates a regression model by using the chemical structures of the low molecular weight compounds selected in S3 and the measured values of the physical property value of the low molecular weight compounds selected in S3 (values obtained by the experiment in S4). The regression model is a model (that is, a model for estimating the “physical property value” from the “chemical structure of low molecular weight compounds”) in which the input is the “chemical structure of the low molecular weight compound” and the output is the “physical property value”.

Hereinafter, in steps 6 and 7, the compound search apparatus 10 calculates an acquisition function by using a Bayesian optimization method.

In step 6 (S6), the compound search apparatus 10 uses the regression model (specifically, a Gaussian process regression model) generated in S5 to calculate an estimate value of a physical property value and a variation of the estimate value from the chemical structure of the low molecular weight compounds other than those in the most diverse group of compounds selected in S3 among the plurality of low molecular weight compounds obtained in S1.

In step 7 (S7), the compound search apparatus 10 calculates an acquisition function from the estimate value of the physical property value calculated in S6 and the variation of the estimate value. Thereafter, the compound search apparatus 10 determines a low molecular weight compound for which the acquisition function is the maximum or minimum (for example, if the acquisition function is expected improvement, the low molecular weight compound having the maximum expected improvement). The chemical structure of the low molecular weight compound having the maximum or minimum acquisition function is displayed on the compound search apparatus 10 or the user terminal 20.

In step 8 (S8), the experimenter carries out an experiment by using the low molecular weight compound determined based on the acquisition function calculated in S7 (for example, if the acquisition function is expected improvement, the low molecular weight compound having the maximum expected improvement). As a result of the experiment, when the objective is achieved (when the measured value of the physical property value meets the target value of the physical property value), the process is ended, and when the objective is not achieved, the process returns to step 5, and the regression model is generated again by adding the value obtained by the experiment in S8, (that is, the compound search apparatus 10 adds the chemical structures of the low molecular weight compounds subjected to the experiment in S8 and the measured values of the physical property value of the low molecular weight compounds subjected to the experiment in S8 (the values obtained by the experiment in S8) to the data set for machine learning and relearns the regression model).

The details of each process will be described below.

### <<Acquisition of a Plurality of Low Molecular Weight Compounds (S1 in FIG. 3)>>

First, the acquisition of a plurality of low molecular weight compounds will be described. The compound search apparatus 10 acquires the chemical structures of a plurality of low molecular weight compounds (candidate compounds that are compounds to be candidates). The following description will be divided into [compounds selected by a person], [compounds extracted from a database], and [compounds designed by a specific algorithm].

### [Compounds Selected by a Person]

For example, the compound search apparatus 10 acquires a plurality of low molecular weight compounds selected by a person (for example, the experimenter) as candidate compounds. In this case, compounds utilizing the knowledge of a person (for example, the experimenter) can be used as candidate compounds.

[Compounds Extracted from the Database]

For example, the compound search apparatus 10 acquires a plurality of low molecular weight compounds extracted from any compound database as candidate compounds. In this case, various compounds in the compound database can be used as candidate compounds.

### [Compounds Designed by a Specific Algorithm]

For example, the compound search apparatus 10 acquires a plurality of low molecular weight compounds designed by a specific algorithm as candidate compounds. For example, the algorithm may be an algorithm that fragments molecules into substructures, an algorithm that fragments molecules into substructures and then connects those substructures, or an algorithm that lists arbitrary substructures and then connects those substructures.

<<Replacement of Chemical Structure with Descriptor (S2 in FIG. 3)>>

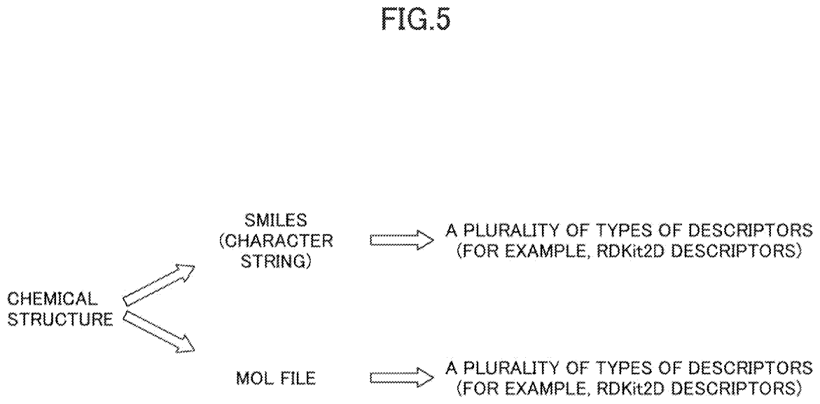

Next, the replacement of chemical structures with descriptors will be described. The compound search apparatus 10 replaces chemical structures of low molecular weight compounds with a plurality of types of descriptors. The descriptors of chemical structures of compounds are those that quantify the characteristics of compounds. The descriptors will be described in detail with reference to FIG. 5.

FIG. 5 is a diagram for explaining the substitution of a chemical structure with a plurality of types of descriptors according to an embodiment of the present disclosure.

For example, the compound search apparatus 10 can convert a chemical structure of a low molecular weight compound into a character string (for example, a character string of SMILES (simplified molecular input line entry system)) and replace the character string with a plurality of types of descriptors. The description method of the chemical structure is not limited to SMILES and may be another description method (for example, a MOL file).

For example, the compound search apparatus 10 can replace the chemical structure of a plurality of low molecular weight compounds with a plurality of types of RDKit2D descriptors.

### <<Selection of the Most Diverse Group of Compounds (S3 in FIG. 3)>>

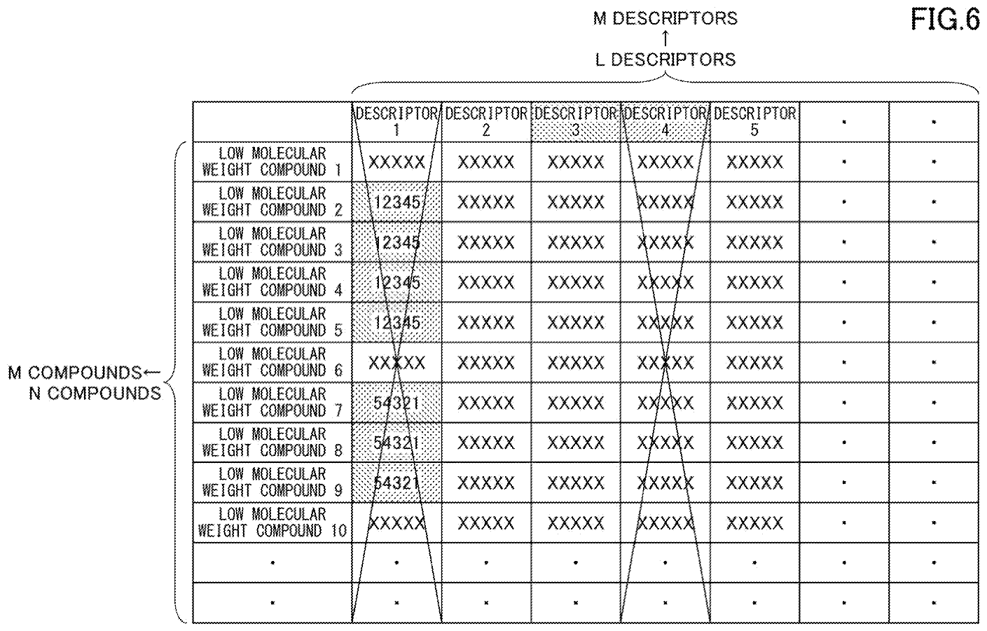

Next, the selection of the most diverse group of compounds will be described. The data set for machine learning is preferably a data set of a diverse group of compounds. If the compounds in the dataset for machine learning were all similar compounds (that is, the descriptors are similar), then the application range of the model generated by machine learning would be small (that is, if a model is generated by using only a certain compound, no other compounds can be estimated). Therefore, the compound search apparatus 10 selects the most diverse group of compounds among a plurality of low molecular weight compounds based on a plurality of types of descriptors by using a D-optimization technique. The description will be described in detail with reference to FIG. 6.

FIG. 6 is a diagram for explaining the selection of the most diverse group of compounds according to an embodiment of the present disclosure. It is assumed that each of a plurality of low molecular weight compounds (low molecular weight compound 1, low molecular weight compound 2, low molecular weight compound 3, low molecular weight compound 4, low molecular weight compound 5, low molecular weight compound 6, low molecular weight compound 7, low molecular weight compound 8, low molecular weight compound 9, low molecular weight compound 10, . . . , low molecular weight compound N in FIG. 6) is replaced by a plurality of types of descriptors (descriptor 1, descriptor 2, descriptor 3, descriptor 4, descriptor 5, . . . , descriptor L of FIG. 6). It is assumed that M low molecular weight compounds (the most diverse group of M compounds) are selected from N low molecular weight compounds (N candidate compounds). It is also assumed that the number of a plurality of types of descriptors is L.

The compound search apparatus 10 can select the most diverse group of compounds from among the plurality of low molecular weight compounds by selecting a combination in which det (X^{T}X) is maximum for the plurality of types of descriptors. The det (X^{T}X) is the D optimization criterion (determinant: det (X^{T}X) is the value calculated from XIX (formula (1) below is a matrix of X^{T}X)).

### [Formula 1]

$$\\ \begin{matrix}\begin{matrix} \\ DESCRIPTOR \\ DESCRIPTOR \\ \\ DESCRIPTOR\end{matrix} \begin{matrix} \\ ( \begin{matrix}{x}_{1}^{{}_{} ( 1 )} & {x}_{1}^{{}_{} ( 2 )} & … & {x}_{1}^{{}_{} ( M )} \\ {x}_{2}^{{}_{} ( 1 )} & {x}_{2}^{{}_{} ( 2 )} & … & {x}_{2}^{{}_{} ( M )} \\ … & … & … & … \\ {x}_{M}^{{}_{} ( 1 )} & {x}_{M}^{{}_{} ( 2 )} & … & {x}_{M}^{{}_{} ( M )}\end{matrix} )\end{matrix} \begin{matrix}DESCRIPTOR & DESCRIPTOR & & DESCRIPTOR \\ {x}_{1}^{{}_{} ( 1 )} & {x}_{2}^{{}_{} ( 1 )} & ⋮ & \\ {x}_{1}^{{}_{} ( 2 )} & {x}_{2}^{{}_{} ( 2 )} & ⋮ & \\ ⋮ & ⋮ & ⋮ & \\ {x}_{1}^{{}_{} ( M )} & {x}_{2}^{{}_{} ( M )} & ⋮ & \end{matrix} \begin{matrix} \\ ( \begin{matrix}{x}_{M}^{{}_{} ( 1 )} \\ {x}_{M}^{{}_{} ( 2 )} \\ ⋮ \\ {x}_{M}^{{}_{} ( M )}\end{matrix} )\end{matrix} & FORMULA ( 1 )\end{matrix}$$

Here, in order to prevent det (X^{T}X)=0, it is essential that the number of the most diverse compound groups (M)≥the number of descriptors. Therefore, the compound search apparatus 10 narrows down the number of descriptors to M. Then, a matrix of N×M is formed. The compound search apparatus 10 selects the combination with the maximum value of det (X^{T}X) among _{N}C_{M} versions of det (X^{T}X). For example, the compound search apparatus 10 may use a genetic algorithm to select the combination with the maximum value of det (X^{T}X) among _{N}C_{M} versions of det (X^{T}X).

<<Narrowing Down to Descriptors with a Large Amount of Information>>

In narrowing down the descriptors, it is preferable that a descriptor having a large amount of information remains. Therefore, the compound search apparatus 10 narrows down the descriptors by using the following method. Hereinafter, the description will be divided into [deletion of descriptors having a high ratio of same values] and [selecting some of highly correlated descriptors].

### [Deletion of Descriptors Having a High Ratio of Same Values]

The compound search apparatus 10 deletes descriptors having a high ratio of the same value (for example, descriptor 1 of FIG. 6). Specifically, the compound search apparatus 10 deletes descriptors for which the ratio of the low molecular weight compounds having the same value to all low molecular weight compounds is larger than a threshold value.

### [Selecting Some of Highly Correlated Descriptors]

The compound search apparatus 10 selects some of descriptors that are highly correlated, with respect to a plurality of types of descriptors (for example, one descriptor (descriptor 4) in a set of highly correlated descriptors (descriptor 3 and descriptor 4 in FIG. 6) is deleted). Specifically, with respect to each of the descriptors in a set of highly correlated descriptors, the compound search apparatus 10 calculates the correlation coefficients with all other descriptors, converts the correlation coefficients into absolute values, and deletes the descriptor whose value of the sum of all absolute values is larger.

The descriptors will now be described. For example, descriptors may include at least one of the molecular weight, the molecular weight calculated for atoms other than hydrogen atoms, the number of valence electrons, the number of atoms other than hydrogen atoms, the sum of nitrogen atoms and “hydrogen bonded to nitrogen atoms” and hydroxyl groups, the sum of nitrogen atoms and oxygen atoms, the number of hydrogen atoms and “atoms other than carbon atoms”, the number of rotatable bonds, the number of rings, the number of radicals, the octanol/water partition coefficient, the number of spiro atoms, the number of bridgehead atoms, the number of aliphatic carbon rings, the number of aliphatic heterocycles, the number of aliphatic rings, the number of aromatic carbons, the number of aromatic heterocycles, the number of aromatic rings, the number of hydrogen acceptor groups, the number of hydrogen donor groups, the number of saturated carbon rings, the number of saturated heterocycles, the number of saturated rings, the number of rings, the number of aliphatic carboxylic acids, the number of aliphatic hydroxyl groups, the number of nitrogen functional groups bonded to aromatic compounds, the number of aromatic carboxylic acids, the number of aromatic nitrogens, the number of aromatic amines, the number of aromatic hydroxyl groups, the number of carboxylic acids, the number of ester groups, the number of carbonyl groups, the number of carbonyl groups excluding carboxylic acids, the number of thiocarbonyl groups, the number of imines, the number of tertiary amines, the number of secondary amines, the number of primary amines, the number of hydroxylamino groups, the number of pyrrole structures, the number of thiol groups, the number of aldehydes, the number of alkyl carbamates, the number of alkyl halides, the number of amides, the number of amidines, the number of anilines, the number of azide groups, the number of barbituric acid structures, the number of benzene rings, the number of bicyclic structures, the number of diazo groups, the number of dihydropyridines, the number of epoxide rings, the number of esters, the number of ether oxygens, the number of furan rings, the number of guanidine groups, the number of halogens, the number of hydrazine groups, the number of imidazole rings, the number of imide groups, the number of isocyanates, the number of isothiocyanates, the number of ketones, the number of beta lactams, the number of cyclic esters, the number of methoxy groups, the number of morpholine rings, the number of nitriles, the number of nitro groups, the number of nitrobenzene structures, the number of non-orthonitrobenzene structures, the number of oxazole rings, the number of phenol structures, the number of phosphate groups, the number of phosphate ester groups, the number of piperidine rings, the number of piperidine rings, the number of primary amides, the number of pyridine rings, the number of quaternary nitrogens, the number of thioethers, the number of sulfonamides, the number of sulfone groups, the number of terminal acetylenes, the number of tetrazole rings, the number of thiazole rings, the number of thiophene rings, or the number of urea structures.

### <<Estimation of Physical Property Value and Calculation of Variation (S6 in FIG. 3)>>

Next, the estimate value of the physical property value and the calculation of the variation of the estimate value will be described.

The compound search apparatus 10 calculates the estimate value of the physical property value of each low molecular weight compound and the variation of the estimate value from the chemical structure of each low molecular weight compound by using a regression model (specifically, a Gaussian process regression model). That is, the compound search apparatus 10 outputs a probability distribution of the physical property value (the estimate value is the average value of the probability distribution, and the variation of the estimate value is the standard deviation of the probability distribution).

### <<Calculation of the Acquisition Function (S7 in FIG. 3)>>

Next, calculation of the acquisition function will be described. The compound search apparatus 10 calculates the acquisition function from the estimate value of the physical property value and the variation of the estimate value. The compound search apparatus 10 can determine the low molecular weight compound for which the acquisition function is the maximum or minimum.

For example, the acquisition function is expected improvement (EI). When the acquisition function is expected improvement (EI), the compound search apparatus 10 can select a compound that has a high possibility of satisfying a desired physical property by determining a low molecular weight compound that has the maximum expected improvement (EI). Note that the acquisition function is not limited to expected improvement (EI). The types of acquisition functions will be described below.

### [Acquisition Function]

The acquisition function may be any of the following four types. EI is the expected improvement, PI is the improvement probability, UCB is the upper confidence limit, and LCB is the lower confidence limit. When the acquisition function is EI, PI, and UCB, the low molecular weight compound with the highest EI, PI, and UCB is determined. When the acquisition function is LCB, the low molecular weight compound with the lowest LCB is determined. EI, PI, UCB, and LCB are calculated by using the following formulas.

### 1. EI (Expected Improvement)

$$\\ EI ( x ) = ( μ ( x ) - {y}_{max} ) Φ ( Z ) + σ ( x ) φ ( Z ) if σ ( x ) > 0$$

$$\\ EI ( x ) = 0 if σ ( x ) = 0$$

Note that Φ is the cumulative density function (the value obtained by integrating the probability density function from −∞ to a specific value), φ is the probability density function, and Z indicates (yₘₐₓ−μ)/σ(x). The notation of u is the average value, and σ is the standard deviation.

### 2. PI (Probability of Improvement)

$$\\ PI ( x ) = {\int }_{ymax}^{∞} N ( f \vert μ ( {x}^{2} ) , σ ( x ) ) df$$

Note that f˜N(f|μ, σ²) is the estimated result by a Gaussian process, and f˜N(f|μ, σ²) indicates that the probability variable f follows a normal distribution with average value μ and variation σ². The notation of μ is the average value and σ is the standard deviation.

### 3. UCB (Upper Confidence Bound)

$$\\ UCB ( x ) = μ ( x ) + k σ ( x )$$

Note that μ(x) is the term corresponding to the exploitation and σ(x) is the term corresponding to the search. The notation of k indicates the degree of the importance placed on the search. The notation of μ is the average value and σ is the standard deviation.

### 4. LCB (Lower Confidence Bound)

$$\\ LCB ( x ) = μ ( x ) - k σ ( x )$$

Note that μ(x) is the term corresponding to the utilization, and σ(x) is the term corresponding to the search. The notation of k indicates the degree of the importance placed on the search. The notation of μ is the average value and σ is the standard deviation.

Thus, by using a Bayesian optimization method, the compound search apparatus 10 can select compounds that are highly likely to satisfy the desired physical property.

### Other Embodiment

The Bayesian optimization applied to simultaneous experiments of multiple compounds will be described below.

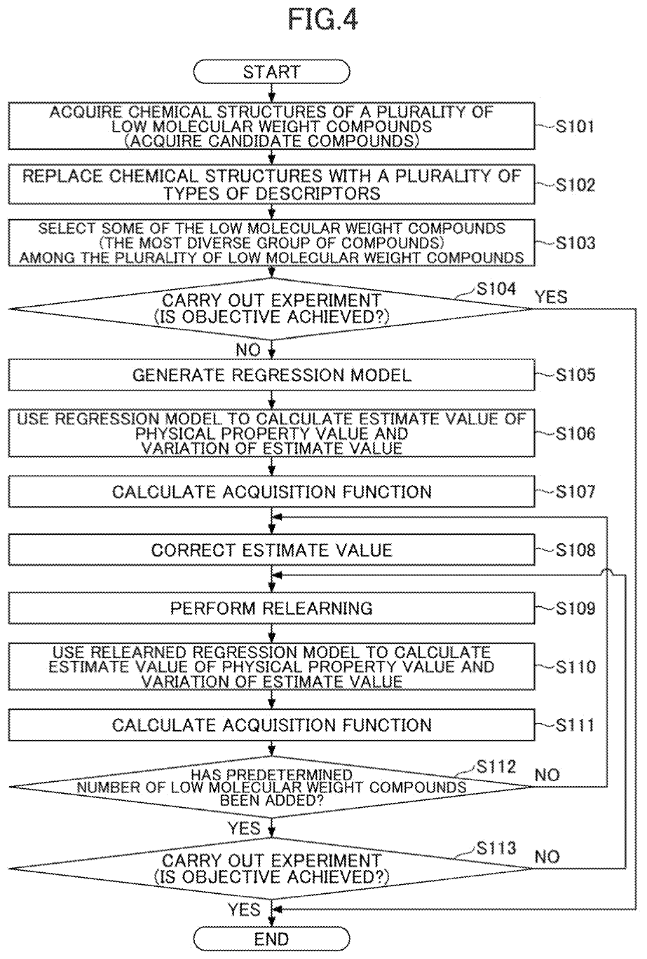

FIG. 4 is a diagram for explaining the overall flow according to an embodiment of the present disclosure. Note that the compound search apparatus 10 acquires a target value (for example, the target value input by the experimenter, etc.) of the physical property value of the low molecular weight compound.

In step 101 (S101), the compound search apparatus 10 acquires the chemical structures of a plurality of low molecular weight compounds. That is, the compound search apparatus 10 acquires candidate compounds that are compounds to be candidates.

In step 102 (S102), the compound search apparatus 10 replaces the chemical structures of the plurality of low molecular weight compounds acquired in S101 with a plurality of types of descriptors (for example, the RDKit2D descriptor).

In step 103 (S103), the compound search apparatus 10 selects some of the low molecular weight compounds (for example, the most diverse group of compounds) among the plurality of low molecular weight compounds acquired in S101 based on the plurality of types of descriptors obtained in S102. Specifically, the compound search apparatus 10 selects the most diverse group of compounds by using a D-optimization technique.

In step 104 (S104), the experimenter performs an experiment by using the most diverse group of compounds selected in S103. As a result of the experiment, when the objective is achieved (if the measured value of the physical property value satisfies the target value of the physical property value), the process is ended, and when the objective is not achieved, the process proceeds to step 105.

In step 105 (S105), the compound search apparatus 10 generates a regression model (specifically, a Gaussian process regression model) by using the most diverse group of compounds selected in S103. Specifically, the compound search apparatus 10 generates a regression model by using the chemical structures of the low molecular weight compounds selected in S103 and the measured values of the physical property value of the low molecular weight compounds selected in S103 (values obtained by the experiment in S104). The regression model is a model (that is, a model for estimating the “physical property value” from the “chemical structure of low molecular weight compounds”) in which the input is the “chemical structure of the low molecular weight compound” and the output is the “physical property value”.

Hereinafter, in steps 106 and 107, the compound search apparatus 10 calculates an acquisition function by using a Bayesian optimization method.

In step 106 (S106), the compound search apparatus 10 calculates the estimate value of the physical property value and the variation of the estimate value from the chemical structure of the low molecular weight compound other than the most diverse group of compounds selected in S103 among the plurality of low molecular weight compounds obtained in S101, by using the regression model (specifically, a Gaussian process regression model) generated in S105.

In step 107 (S107), the compound search apparatus 10 calculates the acquisition function from the estimate value of the physical property value calculated in S106 and the variation of the estimate value. Thereafter, the compound search apparatus 10 determines the low molecular weight compound for which the acquisition function is the maximum or minimum (for example, if the acquisition function is expected improvement, the low molecular weight compound having the maximum expected improvement). The chemical structure of the low molecular weight compound for which the acquisition function is the maximum or minimum is displayed on the compound search apparatus 10 or the user terminal 20.

In step 108 (S108), the compound search apparatus 10 corrects the estimate value (specifically, increases or decreases the estimate value) of the physical property value calculated in S106 of the low molecular weight compound determined in S107. For example, the compound search apparatus 10 corrects the estimate value of the physical property value according to an instruction for correction input by a person (for example, an experimenter). For example, the compound search apparatus 10 corrects the estimate value of the physical property value based on a predetermined rule.

In step 109 (S109), the compound search apparatus 10 adds the estimate value corrected in S108 and generates the regression model again (that is, the compound search apparatus 10 adds the chemical structure of the low molecular weight compound whose estimate value has been corrected in S108 and the estimate value corrected in S108 (estimate value after correction) to the data set for machine learning and relearns the regression model).

In step 110 (S110), the compound search apparatus 10 calculates the estimate value of the physical property value and the variation of the estimate value from the chemical structure of the low molecular weight compounds other than the most diverse group of compounds selected in S103 among the plurality of low molecular weight compounds obtained in S101, by using the regression model (specifically, a Gaussian process regression model) relearned in S109.

In step 111 (S111), the compound search apparatus 10 calculates the acquisition function from the estimate value of the physical property value and the variation of the estimate value calculated in S110. Thereafter, the compound search apparatus 10 determines the low molecular weight compound for which the acquisition function is the maximum or minimum (for example, if the acquisition function is expected improvement, the low molecular weight compound having the maximum expected improvement). The chemical structure of the low molecular weight compound for which the acquisition function is the maximum or minimum is displayed on the compound search apparatus 10 or the user terminal 20.

In the present embodiment, the experimenter carries out an experiment by using the low molecular weight compound determined in S107 and the low molecular weight compound determined in S111. That is, by repeating steps S108 to S112, the low molecular weight compounds to be subjected to the experiment are added.

In step 112 (S112), the compound search apparatus 10 determines whether a predetermined number of low molecular weight compounds (that is, the number of low molecular weight compounds to be subjected to the experiment) have been added. If such a number of low molecular weight compounds have been added, the process proceeds to step 113, and if such a number of low molecular weight compounds have not been added, the process returns to step 108 to correct the estimated physical property value (specifically, increase or decrease the estimate value) calculated in S110 of the low molecular weight compounds determined in step 111.

In step 113 (S113), the experimenter carries out the experiment by using the low molecular weight compounds determined in S107 and the low molecular weight compounds determined in S111. When the objective is achieved as a result of the experiment (when the measured value of the physical property value satisfies the target value of the physical property value), the process is ended, and when the objective is not achieved, the process returns to step 109, and the regression model is generated again by adding the values obtained by the experiment in S113 (that is, the compound search apparatus 10 adds the chemical structure of the low molecular weight compound subjected to the experiment at S113 and the measured physical property value of the low molecular weight compound subjected to the experiment at S113 (the value obtained by the experiment at S113) to the data set for machine learning and relearns the regression model).

According to the first aspect of the present disclosure, it is possible to search for a low molecular weight compound that is highly likely to have the desired physical property.

According to the second aspect of the present disclosure, the low molecular weight compound with the maximum expected improvement can be searched.

According to a third aspect of the present disclosure, it is not necessary to perform experiments beforehand to generate a regression model.

According to a fourth aspect of the present disclosure, it is not necessary to perform experiments in advance to generate a regression model, and it is possible to use a regression model generated by some low molecular weight compounds selected based on a plurality of types of descriptors.

According to a fifth aspect of the present disclosure, it is possible to handle a chemical structure represented by a character string.

According to a sixth aspect of the present disclosure, a chemical structure can be treated numerically.

According to the seventh aspect of the present disclosure, a desired low molecular weight compound can be searched by considering the average value and the standard deviation of the physical property value.

According to an eighth aspect of the present disclosure, candidate compounds can be obtained by various techniques.

According to the ninth aspect of the present disclosure, uncertainty in the estimation of physical property value can be identified.

According to a tenth aspect of the present disclosure, experiments can be performed by using a plurality of low molecular weight compounds at once, rather than one low molecular weight compound at a time.

According to an eleventh aspect of the present disclosure, a diverse group of compounds can be selected from a plurality of low molecular weight compounds.

According to a twelfth aspect of the present disclosure, a descriptor having a large amount of information can be retained.

According to the thirteenth aspect of the present disclosure, a large amount of computations can be accommodated.

According to a fourteenth aspect of the present disclosure, the application range of the regression model can be extended.

Although the embodiments have been described above, it will be understood that various changes in form and details are possible without departing from the object and scope of the claims.

### EXPLANATION OF REFERENCES

1 compound search system
10 compound search apparatus
20 user terminal
30 user
101 control unit
102 ROM
103 RAM
104 auxiliary storage device
105 display device
106 operation device
107 I/F device
108 drive device
109 bus
110 storage medium

What is claimed is:

1. A method executed by a computer, the method comprising:
acquiring a chemical structure of a plurality of low molecular weight compounds;
calculating an estimate value of a physical property value of each of the low molecular weight compounds and a variation of the estimate values, from the acquired chemical structure of each of the low molecular weight compounds, by using a regression model; and
calculating an acquisition function from the estimate values of the physical property value and the variation of the estimate values.

2. The method according to claim 1, further comprising:
determining the low molecular weight compound for which an expected improvement is maximum, in a case where the acquisition function is the expected improvement.

3. The method according to claim 1, further comprising:
selecting at least one of the low molecular weight compounds from among the plurality of the low molecular weight compounds; and
generating the regression model by using the chemical structure of the selected at least one of the low molecular weight compounds and a measured value of the physical property value of the selected at least one of the low molecular weight compounds.

4. The method according to claim 1, further comprising:
replacing the chemical structures of the plurality of the low molecular weight compounds with a plurality of types of descriptors;
selecting at least one of the low molecular weight compounds from among the plurality of the low molecular weight compounds based on the plurality of types of descriptors; and
generating the regression model by using the chemical structure of the selected at least one of the low molecular weight compounds and a measured value of the physical property value of the selected at least one of the low molecular weight compounds.

5. The method according to claim 4, further comprising:
converting the chemical structures of the plurality of the low molecular weight compounds into character strings.

6. The method according to claim 4, further comprising:
replacing the chemical structures of the plurality of the low molecular weight compounds with a plurality of types of RDKit2D descriptors.

7. The method according to claim 1, wherein the estimate value is an average value of a probability distribution, and the variation is a standard deviation of the probability distribution.

8. The method according to claim 1, wherein the plurality of the low molecular weight compounds include at least one of a compound selected by a human, a compound extracted from a database, or a compound designed by a predetermined algorithm.

9. The method according to claim 1, wherein the regression model is a Gaussian process regression model.

10. The method according to claim 1, further comprising:
correcting the estimate value of the physical property value; and
generating the regression model by using the chemical structure of the low molecular weight compound for which the estimate value of the physical property value has been corrected, and the corrected estimate value of the physical property value.

11. A method executed by a computer, the method comprising:
replacing a chemical structure of a plurality of low molecular weight compounds with a plurality of types of descriptors; and
selecting a most diverse group of compounds from among the plurality of the low molecular weight compounds, by selecting a combination in which det (X^{T}X) is maximum for the plurality of types of descriptors.

12. The method according to claim 11, further comprising:
selecting, with respect to the plurality of types of the descriptors, at least one of the descriptors from among highly correlated descriptors, wherein
the most diverse group of compounds is selected by using the selected at least one of the descriptors, and
a number of the selected at least one of the descriptors is less than or equal to a number of the low molecular weight compounds forming the most diverse group of compounds.

13. The method according to claim 11, wherein the most diverse group of compounds is selected by using a genetic algorithm.

14. The method according to claim 11, further comprising:
generating a regression model by using the chemical structure of the low molecular weight compounds forming the most diverse group of compounds and a measured value of a physical property value of the low molecular weight compounds forming the most diverse group.

15. A non-transitory computer-readable recording medium storing a program that causes a computer to execute a process, the process comprising:
acquiring a chemical structure of a plurality of low molecular weight compounds;
calculating an estimate value of a physical property value of each of the low molecular weight compounds and a variation of the estimate values, from the acquired chemical structure of each of the low molecular weight compounds, by using a regression model; and
calculating an acquisition function from the estimate values of the physical property value and the variation of the estimate values.

16. An apparatus comprising:
a processor; and
a memory that includes instructions, which when executed, cause the processor to execute:
acquiring a chemical structure of a plurality of low molecular weight compounds;
calculating an estimate value of a physical property value of each of the low molecular weight compounds and a variation of the estimate values, from the acquired chemical structure of each of the low molecular weight compounds, by using a regression model; and
calculating an acquisition function from the estimate values of the physical property value and the variation of the estimate values.

17. A system comprising:
a server; and
a user terminal, wherein
the server includes:
a processor; and
a memory that includes instructions, which when executed, cause the processor to execute:
acquiring a chemical structure of a plurality of low molecular weight compounds;
calculating an estimate value of a physical property value of each of the low molecular weight compounds and a variation of the estimate values, from the acquired chemical structure of each of the low molecular weight compounds, by using a regression model;
calculating an acquisition function from the estimate values of the physical property value and the variation of the estimate values; and
presenting the acquisition function to the user terminal.
